(19) United States
(12) Patent Application Publication
(10) Pub. No.: US 2020/0261160 A1
(43) Pub. Date: Aug. 20, 2020

(54) ROBOTIC SURGICAL SYSTEMS AND METHODS AND COMPUTER-READABLE MEDIA FOR CONTROLLING THEM

(71) Applicant: Covidien LP, Mansfield, MA (US)

(72) Inventors: William Peine, Ashland, MA (US); Albert Dvornik, Somerville, MA (US); Jared Farlow, Los Angeles, CA (US); Robert W. Pierce, Franklin, MA (US); Robert L. Stephenson, Carlsbad, CA (US)

(21) Appl. No.: 16/644,557

(22) PCT Filed: Aug. 29, 2018
(86) PCT No.: PCT/US2018/048475

§ 371 Date: Mar. 5, 2020

Related U.S. Application Data
(60) Provisional application No. 62/554,093, filed on Sep. 5, 2017.

(51) Int. Cl. A61B 34/20; A61B 34/37; A61B 34/00; A61B 90/00; A61B 90/50

(52) U.S. Cl. CPC A61B 34/20; A61B 34/37; A61B 34/74; A61B 34/76; A61B 2090/3937; A61B 90/361; A61B 90/50; A61B 2034/2055; A61B 2034/742; A61B 90/39

(57) ABSTRACT

Robotic surgical systems are operated in a manner that improves user experience and user control. The robotic surgical system systems are configured to have a carrying mode, a camera reposition mode, and/or a targeting mode, one or more of which is selectable by the user.

### BACKGROUND

Robotic surgical systems are increasingly being used in minimally invasive surgical procedures. Typically, robotic surgical systems include a clinician console located remote from one or more robotic arms to which surgical instruments and/or cameras are coupled. For example, the clinician console may be located on another side of the operating room from the robotic arms, in another room, or in another building, and includes input handles and/or other input devices to be actuated by a clinician. Signals, based on the actuation of the input handles, are communicated to a central controller, which translates the signals into commands for manipulating the robotic arms and/or the surgical instruments coupled thereto, for example, within a surgical site.

In addition to input handles and input devices, the clinician console includes a display. The display provides a view of the surgical site by displaying images captured by the cameras attached to one or more of the robotic arms. In order to position and/or re-position the cameras within the surgical site, the clinician may dissociate actuation of the input handles from the surgical instruments and associate actuation of the input handles with the camera. As a result, when the clinician actuates the input handles, signals based on the actuation are translated into commands to realize a corresponding movement of the cameras. Although current robotic surgical systems are adequate, they may be improved.

### SUMMARY

The present disclosure provides improved robotic surgical systems, and also provides improved methods and computer-readable media for controlling robotic surgical systems.

In an aspect of the present disclosure, a robotic surgical system includes a robotic arm including a surgical instrument, a patient image capture device configured to capture images of a surgical site, and a console. The console includes a display for displaying the captured images of the surgical site, an input handle, and an input device configured to be actuated and to provide a signal based on the actuation for causing the robotic surgical system to enter or exit a camera reposition mode. A controller is coupled to the robotic arm, the patient image capture device, and the console. The controller includes a processor, and memory coupled to the processor. The memory has instructions stored thereon that, when executed by the processor, cause the controller, in response to the signal received based on actuation of the input device, to cause the robotic surgical system to enter the camera reposition mode. When the robotic surgical system is in the camera reposition mode, the controller disassociates actuation of the input handle from movement of the robotic arm, and tracks a position of a user's head.

In another aspect of the present disclosure, the input device includes a button on the input handle.

In another aspect of the present disclosure, the input device includes a foot pedal.

In still another aspect of the present disclosure, the memory has further instructions stored thereon that, when executed by the processor, cause the controller to enter the camera reposition mode in response to receiving a first signal based on a first actuation of the foot pedal, and exit the camera reposition mode in response to receiving a second signal based on a second actuation of the foot pedal within a predetermined time of the receiving of the first signal.

In still another aspect of the present disclosure, the memory has further instructions stored thereon that, when executed by the processor, cause the controller to enter the camera reposition mode in response to receiving a signal indicating that the foot pedal has been depressed, and exit the camera reposition mode in response to receiving a signal indicating that the foot pedal has been released.

In another aspect of the present disclosure, the robotic surgical system also includes a user image capture device configured to capture images of the user for tracking a motion of the user's head.

In still another aspect of the present disclosure, the memory has further instructions stored thereon that, when executed by the processor, cause the controller to detect the position of the user's head from images of the user captured by the user image capture device, determine from the captured images of the user whether a left or right tilt of the user's head has occurred, and in response to a determination that the tilt of the user's head is a left tilt or a right tilt, cause the patient image capture device to correspondingly pan to the left or to the right.

In still another aspect of the present disclosure, the memory has further instructions stored thereon that, when executed by the processor, cause the controller to detect the position of the user's head from the captured images of the user, determine whether a roll of the user's head has occurred, and in response to the determination of the roll of the user's head, cause the patient image capture device to roll in a motion corresponding to the roll of the user's head.

In another aspect of the present disclosure, the memory has further instructions stored thereon that, when executed by the processor, cause the controller, when in the camera reposition mode, to increase a scaling factor between a signal received based on actuation of the input handle and an output movement by the surgical instrument.

In another aspect of the present disclosure, the memory has further instructions stored thereon that, when executed by the processor, cause the controller, when the robotic surgical system is in the camera reposition mode, to provide at least one of a force feedback signal or a torque feedback signal to reduce an output movement by the surgical instrument corresponding to the signal received based on the actuation of the input handle to prevent manipulation of the input handle from moving the surgical instrument.

According to an aspect of the present disclosure, a method of controlling a robotic surgical system includes generating at least one signal, based on actuation of an input device of the robotic surgical system, the at least one signal causing the robotic surgical system to enter or exit a camera reposition mode. In response to the at least one signal, the robotic surgical system enters the camera reposition mode. When the robotic surgical system is in the camera reposition mode, actuation of an input handle of the robotic surgical system is disassociated from movement of a robotic arm of the robotic surgical system, and a position of a user's head is tracked by a user image capture device.

In another aspect of the present disclosure, the input device includes a foot pedal. The method further includes entering the camera reposition mode in response to receiving a first signal generated by a first actuation of the foot pedal, and exiting the camera reposition mode in response to receiving a second signal generated by a second actuation of the foot pedal within a predetermined time of generating the first signal.

In another aspect of the present disclosure, the input device includes a foot pedal, and the method further includes entering the camera reposition mode in response to a generated signal indicating that the foot pedal has been depressed, and exiting the camera reposition mode, in response to a generated signal indicating that the foot pedal has been released.

In another aspect of the present disclosure, the method further includes capturing images of the user's head. A determination is made as to whether a left or right tilt in the position of the user's head has occurred. In response to a determination that the tilt of the user's head is a left tilt or a right tilt, a patient image capture device of the robotic surgical system correspondingly pans to the left or to the right.

In another aspect of the present disclosure, the method further includes capturing images of the user's head. A determination is made as to whether a roll of the user's head has occurred. In response to a determination that a roll of the user's head has occurred, a patient image capture device of the robotic surgical system rolls in a motion corresponding to the roll of the user's head.

In another aspect of the present disclosure, the method further includes, when in the camera reposition mode, increasing a scaling factor between the at least one signal received based on actuation of the input handle and an output movement by a surgical instrument of the robotic surgical system.

In another aspect of the present disclosure, the method further includes, when in the camera reposition mode, providing at least one of a force feedback signal or a torque feedback signal to reduce an output to the surgical instrument corresponding to the signal received based on the actuation of the input handle to prevent actuation of the input handle from moving a surgical instrument of the robotic surgical system.

According to still another aspect of the present disclosure, a non-transitory computer-readable medium has instructions stored thereon which, when executed by a processor, cause the processor to perform a method for controlling a robotic surgical system. The method includes receiving at least one signal based on actuation of an input device of the robotic surgical system, the at least one signal causing the robotic surgical system to enter or exit a camera reposition mode, and in response to receipt of the at least one signal, causing the robotic surgical system to enter the camera reposition mode. When the robotic surgical system is in the camera reposition mode, actuation of an input handle of the robotic surgical system is disassociated from movement of a robotic arm of the robotic surgical system, and a position of a user's head is tracked by a user image capture device.

In another aspect of the present disclosure, the input device includes a foot pedal. The method further includes entering the camera reposition mode in response to receiving a first signal based on a first actuation of the foot pedal, and exiting the camera reposition mode in response to receiving a second signal based on a second actuation of the foot pedal within a predetermined time of the receiving of the first signal.

In another aspect of the present disclosure, where the input device includes a foot pedal, and the method further includes entering the camera reposition mode in response to receiving a signal indicating that the foot pedal has been depressed, and exiting the camera reposition mode in response to receiving a signal indicating that the foot pedal has been released.

In another aspect of the present disclosure, the method further includes determining whether a left tilt or a right tilt in the position of the user's head has occurred based on captured images from the user image capture device, and in response to a determination that the tilt of the user's head is a left tilt or a right tilt, causing a patient image capture device of the robotic surgical system to correspondingly pan to the left or to the right.

In another aspect of the present disclosure, the method further includes determining whether a roll of the user's head has occurred based on captured images from the user image capture device, and in response to a determination that a roll of the user's head has occurred, causing a patient image capture device of the robotic surgical system to roll in a motion corresponding to the roll of the user's head. In still another aspect of the present disclosure, when in the camera reposition mode, a scaling factor is increased between the at least one signal received based on actuation of the input handle and an output movement by a surgical instrument of the robotic surgical system.

In another aspect of the present disclosure, the method further includes, when in the camera reposition mode, providing at least one of a force feedback signal or a torque feedback signal to reduce an output to a surgical instrument of the robotic surgical system corresponding to the at least one signal received based on the actuation of the input handle to prevent actuation of the input handle from moving the surgical instrument.

According to still another aspect of the present disclosure, a robotic surgical system, includes a robotic arm including a surgical instrument, a patient image capture device having an adjustable field of view and being configured to capture images of a surgical site, and a console. The console includes a display for displaying the captured images of the surgical site, an input handle, and an input device. The input device is configured to be actuated and to provide a signal based on the actuation for causing the robotic surgical system to enter or exit a carrying mode. A controller is coupled to the robotic arm, the patient image capture device, and the console. The controller includes a processor, and memory coupled to the processor. The method has instructions stored thereon that, when executed by the processor, cause the controller to receive captured images of the surgical site, receive a signal based on actuation of the input handle to move the surgical instrument, receive a signal based on actuation of the input device; and in response to the signal received based on actuation of the input device, cause the robotic surgical system to enter the carrying mode. The carrying mode includes detecting a surgical instrument in the captured images of the surgical site, determining whether the surgical instrument is in a field of view of the patient image capture device, in response to a determination that the surgical instrument is not within the field of view of the patient image capture device, causing the patient image capture device to adjust the field of view, and in response to a determination that the surgical instrument is within the field of view of the patient image capture device, determining whether the surgical instrument is moving over time in the captured images.

In another aspect of the present disclosure, the memory has further instructions stored thereon that, when executed by the processor, cause the controller, in response to the determination that the surgical instrument is moving, to adjust a pose of the patient image capture device.

According to another aspect of the present disclosure, a method of controlling a robotic surgical system includes receiving a signal based on actuation of an input device of the robotic surgical system. The robotic surgical system includes a robotic arm including a surgical instrument coupled thereto, a patient image capture device having an adjustable field of view and being configured to capture images of a surgical site, and a console. The console includes a display for displaying the captured images of the surgical site, and an input handle. The input device is configured to be actuated and to provide a signal based on the actuation for causing the robotic surgical system to enter or exit a carrying mode. The method includes receiving captured images of the surgical site, receiving a signal based on actuation of the input handle to move the surgical instrument, receiving a signal based on actuation of the input device, and in response to the signal received based on actuation of the input device, cause the robotic surgical system to enter the carrying mode. The carrying mode includes detecting a surgical instrument in the captured images of the surgical site, determining whether the surgical instrument is in a field of view of the patient image capture device, in response to a determination that the surgical instrument is not within the field of view of the patient image capture device, causing the patient image capture device to adjust the field of view, and in response to a determination that the surgical instrument is within the field of view of the patient image capture device, determining whether the surgical instrument is moving over time in the captured images.

In another aspect of the present disclosure, in response to the determination that the surgical instrument is moving, a pose of the patient image capture device is adjusted.

According to still another aspect of the present disclosure, a non-transitory computer-readable medium includes instructions stored thereon, which when executed by a processor, cause the processor to perform a method for controlling a robotic surgical system. The method includes receiving a signal based on actuation of an input device of the robotic surgical system. The robotic surgical system includes a robotic arm including a surgical instrument coupled thereto, a patient image capture device having an adjustable field of view and being configured to capture images of a surgical site, and a console including a display for displaying the captured images of the surgical site and an input handle. The input device is configured to be actuated and to provide a signal based on the actuation for causing the robotic surgical system to enter or exit a carrying mode. The method also includes receiving captured images of the surgical site, receiving a signal based on actuation of the input handle to move the surgical instrument; receiving a signal based on actuation of the input device, and in response to the signal received based on actuation of the input device, causing the robotic surgical system to enter the carrying mode. The carrying mode includes detecting a surgical instrument in the captured images of the surgical site, determining whether the surgical instrument is in a field of view of the patient image capture device, in response to a determination that the surgical instrument is not within the field of view of the patient image capture device, causing the patient image capture device to adjust the field of view, and in response to a determination that the surgical instrument is within the field of view of the patient image capture device, determining whether the surgical instrument is moving over time in the captured images.

In another aspect of the present disclosure, the method further includes, in response to the determination that the surgical instrument is moving, adjusting a pose of the patient image capture device.

According to another aspect, a robotic surgical system includes a patient image capture device having an adjustable field of view and being configured to capture images of a surgical site, a console, and a user image capture device configured to capture images of a user. The console includes a display for displaying the captured images of the surgical site, an input handle, and one or more input devices, wherein a first input device of the one or more input devices is configured to be actuated and to provide a signal based on the actuation for causing the robotic surgical system to enter or exit a targeting mode. A controller is coupled to the patient image capture device, the console, and the user image capture device. The controller including a processor and memory coupled to the processor. The memory has instructions stored thereon that, when executed by the processor, cause the controller to track a position of the user's head from the captured images of the user, receive a signal based on actuation of the first input device, and in response to the signal received based on actuation of the first input device, cause the robotic surgical system to enter the targeting mode. The targeting mode includes causing an icon to be displayed on the display, detecting an initial position of the user's head, determining whether a change has occurred in the position of the user's head from the initial position of the user's head, and in response to a determination that a change has occurred in the position of the user's head, determining whether the change is a velocity change. In response to a determination that the change is a velocity change, a size of the displayed icon is increased to correspond with a positive velocity change or the size of the displayed icon is decreased to correspond with a negative velocity change.

In another aspect of the present disclosure, a second input device of the one or more input devices is configured to be actuated to indicate a confirmation to provide a command to the patient image capture device. The memory has stored thereon further instructions which, when executed by the one or more processors, cause the controller to receive a signal based on actuation of the second input device. In response to the signal received based on actuation of the second input device and the determination that the change in velocity is a negative velocity change, the patient image capture device adjusts from an initial field of view to a first adjusted field of view larger than the initial field of view. In response to the signal received based on actuation of the second input device and the determination that the change in velocity is a positive velocity change, the patient image capture device adjusts from the initial field of view to a second adjusted field of view smaller than the initial field of view.

In another aspect of the present disclosure, wherein the memory has stored thereon further instructions which, when executed by the processor, cause the controller, in response to a determination that a change has occurred in the position of the user's head, to determine whether the change indicates a head roll motion of the user. In response to a determination that the change indicates a head roll motion of the user, the displayed icon rotates in a manner corresponding to the head roll motion of the user. In still another aspect of the present disclosure, a second input device of the one or more input devices is configured to be actuated to indicate a confirmation to provide a command to the patient image capture device. The memory has stored thereon further instructions which, when executed by the processor, cause the controller to receive a signal based on actuation of the second input device, and in response to the signal received input based on actuation of the second input device and the determination that the change indicates a head roll motion of the user, cause the patient image capture device to rotate in a manner corresponding to the head roll motion of the user.

In another aspect of the present disclosure, the memory has stored thereon further instructions which, when executed by the processor, cause the controller, in response to a determination that a change has occurred in the position of the user's head, to determine whether the change indicates a head nod motion. Additionally, in response to a determination that the change indicates a head nod motion of the user, the displayed icon is moved in a direction corresponding to the head nod motion. In still another aspect of the present disclosure, a second input device of the one or more input devices is configured to be actuated to indicate a confirmation to provide a command to the patient image capture device. The memory has stored thereon further instructions, which when executed by the processor, cause the controller to receive a signal based on actuation of the second input device. In response to the signal received based on actuation of the second input device and to a determination that the change indicates a head nod motion of the user, a pose of the patient image capture device is adjusted in a manner corresponding to the head nod motion of the user.

In another aspect of the present disclosure, the memory has stored thereon further instructions which, when executed by the processor, cause the controller, in response to a determination that a change has occurred in the position of the user's head, to determine whether the change indicates a head tilt motion. In response to a determination that the change indicates a head tilt motion of the user, the displayed icon is moved across the image in a direction corresponding to the head tilt motion. In still another aspect of the present disclosure, a second input device of the one or more input devices is configured to be actuated to indicate a confirmation to provide a command to the patient image capture device. The memory has stored thereon further instructions which, when executed by the processor, cause the controller to receive a signal based on actuation of the second input device. In response to the signal received based on actuation of the second input device and to a determination that the change indicates that the head tilt motion is a left tilt motion, the patient image capture device performs a panning motion in a corresponding left direction, and in response to the signal received based on actuation of the second input device and to a determination that the change indicates that the head tilt motion is a right tilt motion, the patient image capture device performs a panning motion in a corresponding right direction.

In another aspect of the present disclosure, the one or more input devices includes a button and a foot pedal.

According to still another aspect of the present disclosure, a method of controlling a robotic surgical system includes tracking a position of the user's head from images of a user captured by a user image capture device. The method also includes receiving a signal based on actuation of a first input device of the robotic surgical system including a patient image capture device having a field of view and being configured to capture images of a surgical site, a console including a display for displaying images from the patient image capture device of the surgical site, an input handle, and one or more input devices including the first input device, wherein the first input device of the one or more input devices is configured to provide a signal for the robotic surgical system to enter or exit a targeting mode. In response to the signal received based on the actuation of the first input device, causing the robotic surgical system to enter the targeting mode. The targeting mode includes causing an icon to be displayed on the display, detecting an initial position of the user's head, and determining whether a change has occurred in the position of the user's head from the initial position of the user's head. Additionally, in response to a determination that a change has occurred in the position of the user's head, a determination is made as to whether the change is a velocity change. In response to a determination that the change is a velocity change, a size of the displayed icon is increased to correspond with a positive velocity change or the size of the displayed icon is decreased to correspond with a negative velocity change.

In another aspect of the present disclosure, the method further includes receiving a signal based on actuation of the second input device. In response to the signal received based on actuation of the second input device and the determination that the change in velocity is a negative velocity change, the patient image capture device is adjusted from an initial field of view to a first adjusted field of view larger than the initial field of view. In response to the signal received based on actuation of the second input device and the determination that the change in velocity is a positive velocity change, the patient image capture device is adjusted from the initial field of view to a second adjusted field of view smaller than the initial field of view.

In another aspect of the present disclosure, the method further includes, in response to a determination that a change has occurred in the position of the user's head, determining whether the change indicates a head roll motion of the user. Additionally, in response to a determination that the change indicates a head roll motion of the user, the displayed icon rotates in a manner corresponding to the head roll motion of the user.

In another aspect of the present disclosure, the method further includes receiving a signal based on actuation of from a second input device. In response to the signal received based on actuation of the second input device and the determination that the change indicates a head roll motion of the user, causing the patient image capture device to rotate in a manner corresponding to the head roll motion of the user.

In another aspect of the present disclosure, the method further includes, in response to a determination that a change has occurred in the position of the user's head, determining whether the change indicates a head nod motion, and in response to a determination that the change indicates a head nod motion of the user, moving the displayed icon in a direction corresponding to the head nod motion. In still another aspect of the present disclosure, the method also includes receiving a signal based on actuation of a second input device, and in response to the signal received based on actuation of the second input device and to a determination that the change indicates a head nod motion of the user, adjusting a pose of the patient image capture device in a manner corresponding to the head nod motion of the user.

In another aspect of the present disclosure, the method further includes, in response to a determination that a change has occurred in the position of the user's head, determining whether the change indicates a head tilt motion, and in response to a determination that the change indicates a head tilt motion of the user, moving the displayed icon across the image in a direction corresponding to the head tilt motion. In still another aspect of the present disclosure, a signal is received based on actuation of a second input device, and in response to the signal received based on actuation of the second input device and to a determination that the change indicates that the head tilt motion is a left tilt motion, the patient image capture device performs a panning motion in a corresponding left direction. In response to the signal received based on actuation of the second input device and to a determination that the change indicates that the head tilt motion is a right tilt motion, the patient image capture device performs a panning motion in a corresponding right direction.

According to still another aspect of the present disclosure, a non-transitory computer-readable medium includes instructions stored thereon, which when executed by a processor, cause the processor to perform a method for controlling a robotic surgical system. The method includes receiving a signal based on actuation of a first input device of the robotic surgical system. The robotic surgical system includes a patient image capture device having a field of view and being configured to capture images of a surgical site, a console including a display for displaying images from the patient image capture device of the surgical site, an input handle, and one or more input devices. The first input device of the one or more input devices is configured to provide a signal for the robotic surgical system to enter or exit a targeting mode, and a user image capture device configured to capture images of a user. The method also includes tracking a position of the user's head from the captured images of the user, and in response to the signal received based on actuation of the first input device, causing the robotic surgical system to enter the targeting mode. The targeting mode includes causing an icon to be displayed on the display, detecting an initial position of the user's head, determining whether a change has occurred in the position of the user's head from the initial position of the user's head, and in response to a determination that a change has occurred in the position of the user's head, determining whether the change is a velocity change. In response to a determination that the change is a velocity change, a size of the displayed icon is increased to correspond with a positive velocity change or the size of the displayed icon is decreased to correspond with a negative velocity change.

In another aspect of the present disclosure, the method further includes receiving a signal based on actuation of the second input device, in response to the signal received based on actuation of the second input device and the determination that the change in velocity is a negative velocity change, causing the patient image capture device to adjust from an initial field of view to a first adjusted field of view larger than the initial field of view, and in response to the signal received based on actuation of the second input device and the determination that the change in velocity is a positive velocity change, causing the patient image capture device to adjust from the initial field of view to a second adjusted field of view smaller than the initial field of view.

In another aspect of the present disclosure, the method further includes in response to a determination that a change has occurred in the position of the user's head, determining whether the change indicates a head roll motion of the user, and in response to a determination that the change indicates a head roll motion of the user, rotating the displayed icon in a manner corresponding to the head roll motion of the user.

In another aspect of the present disclosure, the method further includes receiving a signal based on actuation of a second input device, and in response to the signal received based on actuation of the second input device and the determination that the change indicates a head roll motion of the user, causing the patient image capture device to rotate in a manner corresponding to the head roll motion of the user.

In another aspect of the present disclosure, the method further includes in response to a determination that a change has occurred in the position of the user's head, determining whether the change indicates a head nod motion, and in response to a determination that the change indicates a head nod motion of the user, moving the displayed icon in a direction corresponding to the head nod motion. In still another aspect of the present disclosure, the method further includes receiving a signal based on actuation of a second input device, and in response to the signal received based on actuation of the second input device and to a determination that the change indicates a head nod motion of the user, adjusting a pose of the patient image capture device in a manner corresponding to the head nod motion of the user.

In another aspect of the present disclosure, the method further includes in response to a determination that a change has occurred in the position of the user's head, determining whether the change indicates a head tilt motion, and in response to a determination that the change indicates a head tilt motion of the user, moving the displayed icon across the image in a direction corresponding to the head tilt motion. In still another aspect of the present disclosure, the method further includes receiving a signal based on actuation of a second input device. In response to the signal received based on actuation of the second input device and to a determination that the change indicates that the head tilt motion is a left tilt motion, the patient image capture device performs a panning motion in a corresponding left direction, and in response to the signal received based on actuation of the second input device and to a determination that the change indicates that the head tilt motion is a right tilt motion, the patient image capture device performs a panning motion in a corresponding right direction.

According to another aspect of the present disclosure, a robotic surgical system includes a robotic arm including a surgical instrument, a patient image capture device configured to capture images of a surgical site, and a console. The console includes a display for displaying the captured images of the surgical site, an input handle, a first input device configured to be actuated and to provide a signal based on the actuation for causing the robotic surgical system to enter or exit a carrying mode, and a second input device configured to be actuated and to provide a signal based on the actuation for causing the robotic surgical system to enter or exit a camera reposition mode. A controller is coupled to the robotic arm, the patient image capture device, and the console. The controller includes a processor and memory coupled to the processor. The memory has instructions stored thereon that, when executed by the processor, cause the controller, in response to a signal received based on actuation of the first input device, to cause the robotic surgical system to enter the carrying mode. The carrying mode includes tracking a position of the surgical instrument within the initial field of view from the captured images of the surgical site over a period of time, comparing a tracked position of the surgical instrument at a first time with a tracked position of the surgical instrument within the initial field of view at a second time, and determining whether a distance between the tracked positions at the first time and the second time is greater than a predetermined threshold distance. In response to a determination that the distance between the tracked positions is greater than the predetermined threshold distance, a determination is made as to whether the tracked position of the surgical instrument at the second time is within a predetermined distance from an edge of an initial field of view of the patient image capture device. In response to a determination that the tracked position at the second time is not within a predetermined distance from an edge of the initial field of view of the patient image capture device, a pose of the patient image capture device is adjusted to correspond to the tracked position at the second time, and in response to a determination that the tracked position at the second time is within a predetermined distance from an edge of the initial field of view of the patient image capture device, the initial field of view of the patient image capture device is increased to an adjusted field of view greater than the initial field of view. In response to a signal received based on actuation of the second input device, cause the robotic surgical system to enter the camera reposition mode. The camera reposition mode includes disassociating actuation of the input handle from movement of the robotic arm, and tracking a position of a user's head.

In another aspect of the present disclosure, the robotic surgical system further includes a third input device configured to provide a signal for the robotic surgical system to enter or exit a targeting mode. The memory has further instructions stored thereon that, when executed by the processor, cause the controller to, when in the camera reposition mode, receive a signal based on actuation of the third input device, and in response to the signal received based on actuation of the third input device, enter the targeting mode. The targeting mode includes tracking the position of the user's head from the captured images of the user, causing an icon to be displayed on the display, detecting an initial position of the user's head, determining whether a change has occurred in the position of the user's head from the initial position of the user's head, and in response to a determination that a change has occurred in the position of the user's head, determining whether the change is a velocity change. In response to a determination that the change is a velocity change, a size of the displayed icon is increased to correspond with a positive velocity change or the size of the displayed icon is decreased to correspond with a negative velocity change.

Any of the above aspects and embodiments of the present disclosure may be combined without departing from the scope of the present disclosure.

### BRIEF DESCRIPTION OF THE DRAWINGS

Various aspects and features of the present disclosure are described hereinbelow with references to the drawings, wherein:

FIG. 1 is a simplified diagram of a robotic surgical system, in accordance with an embodiment of the present disclosure;

FIG. 2 is a block diagram of a system architecture of the robotic surgical system of FIG. 1, in accordance with an embodiment of the present disclosure;

FIG. 3 is a flow diagram of a method of controlling a robotic surgical system, in accordance with an embodiment of the present disclosure;

FIG. 4 is a flow diagram of a method of operating the robotic surgical system in a carrying mode, if selected during the performance of the method of FIG. 3, in accordance with an embodiment of the present disclosure;

FIG. 5 is a flow diagram of a method of operating the robotic surgical system in a camera reposition mode, if selected during the performance of the method of FIG. 3, in accordance with an embodiment of the present disclosure;

FIG. 6 is a flow diagram of a method of performing head tracking, if a targeting mode is not selected during the performance of the method of FIG. 3, in accordance with an embodiment of the present disclosure; and

FIG. 7 is a flow diagram of a method of performing head tracking, if a targeting mode is selected during the performance of the method of FIG. 3, in accordance with an embodiment of the present disclosure.

### DETAILED DESCRIPTION

Embodiments of the present disclosure are now described in detail with reference to the drawings in which like reference numerals designate identical or corresponding elements in each of the several views. As used herein, the terms “user” and “clinician” refer to a doctor, a nurse, or any other care provider and may include support personnel. Throughout this description, the term “proximal” refers to the portion of the device or component thereof that is farthest from the patient and the term “distal” refers to the portion of the device or component thereof that is closest to the patient.

The present disclosure is directed to robotic surgical systems, methods, and computer-readable media for controlling the robotic surgical systems in a manner that improves user experience and user control. In an embodiment, the robotic surgical system is configured to be operable in one or more modes selectable by the user via a single or multiple input devices. For example, the robotic surgical system may be configured such that a single input device permits the user to toggle the system between on and off positions to turn a particular mode on or off. In another example, the system is configured to be operated in more than one mode, and different input devices are each associated with a different mode. In this regard, two of the modes operate concurrently, in an embodiment. In another embodiment, operation in a mode may not be selected, unless the system is operating in a prerequisite mode. The robotic surgical system may be configured to operate in one or more of a carrying mode, a camera reposition mode, and/or a targeting mode.

The carrying mode, if selected, causes the patient image capture device to follow a surgical instrument in a surgical site without user input until the user deselects the mode. More particularly, selection of the carrying mode permits a controller of the robotic surgical system to detect a presence of the surgical instrument from images captured from the patient image capture device disposed at a surgical site. The movement of the detected surgical instrument is tracked from the captured images. In response to determinations that the movement is greater than a predetermined threshold distance and that the movement is not approaching an edge of a field of view of the patient image capture device, the patient image capture device is adjusted in a manner corresponding to the movement of the detected surgical instrument. In response to determinations that the movement is greater than a predetermined threshold distance and that the movement is approaching an edge of a field of view of the patient image capture device, a focal length of a lens within the patient image capture device is adjusted (for example, commands are provided to cause the patient image capture device to zoom out) to expand the field of view such that captured images continue to include the surgical instrument.

The camera reposition mode, if selected, prevents user inputs to the input handles from being translated to the robotic arms, and hence, the surgical instrument. As a result, the user may adjust the positions of the input handles at the console without repositioning the robotic arms and/or surgical instruments. Additionally, the patient image capture device can be repositioned, with head tracking being used to drive the movement of the patient image capture device.

While in the camera reposition mode, the targeting mode may be selected. The targeting mode, if selected, provides the user with greater control of the patient image capture device during head tracking. For example, when in the targeting mode, the system displays an icon concurrently with the images captured by the patient image capture device, for example, by superimposing the icon over the images. If the system determines that the user moves closer to or further away from a fixed location, such as the user image capture device, the displayed icon correspondingly increases or decreases in size. If a head roll is detected, the displayed icon correspondingly rotates. If a head tilt is detected either to the left or right, the displayed icon correspondingly moves to the left or right. If a head nod is detected, the displayed icon correspondingly moves up or down on the display. Concurrently with or after one or more of the tracked movements of the user's head, the user may actuate an input device of the robotic surgical system, via, for example, a button, which provides signals to cause the patient image capture device to move according to the tracked movements of the user's head. In another embodiment, the robotic surgical system determines and then stores an original location of the patient image capture device within the surgical site, and the user, using a different input device, can make a selection to cause the patient image capture device to return to the original location.

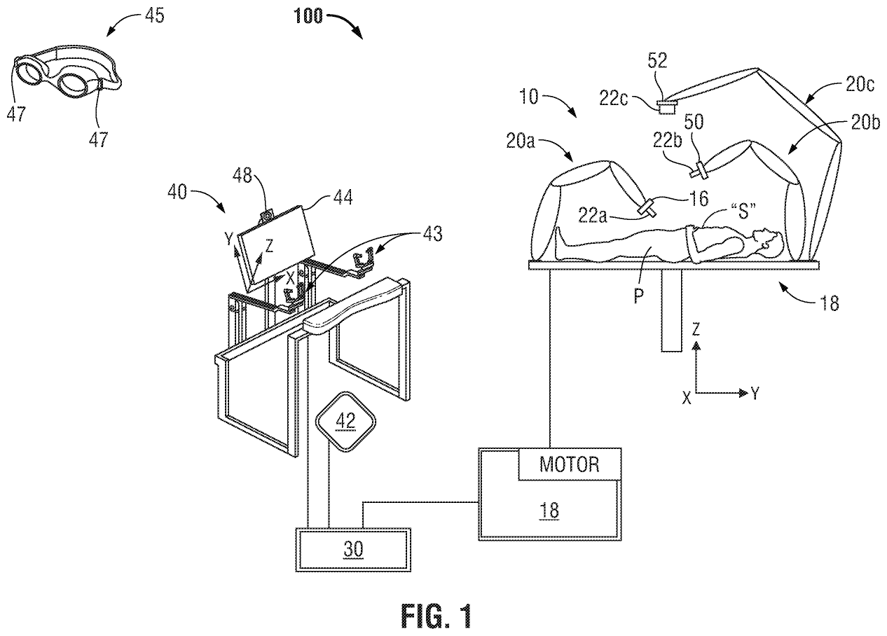

With reference to FIG. 1, a robotic surgical system 100 on which one or more of the modes, which are discussed in greater detail below, may be implemented is provided in accordance with an embodiment of the present disclosure. The robotic surgical system 100 generally includes a surgical robot 10, a robot base 18, a plurality of image capture devices 48, 50, 52, a console 40, and a controller 30. The surgical robot 10 has one or more robotic arms 20a, 20b, 20c which may be in the form of linkages. In an embodiment, one or more of the robotic arms 20a, 20b, 20c, for example, arm 20b, may have a surgical instrument 16 interchangeably fastened to its distal end 22. In another embodiment, one or more of the robotic arms 20a, 20b, 20c may have an image capture device 50, 52 attached thereto. For example, robotic arm 20a may include a patient image capture device 52, and/or robotic arm 20c may include an image capture device 50. Each of the robotic arms 20a, 20b, 20c is moveable about a surgical site “S” around a patient “P.”

Each console 40 communicates with the robot base 18 through the controller 30 and includes a display device 44 which is configured to display images. Although a single robot base 18 is shown, each of the arms 20a, 20b, 20c has a corresponding base in other embodiments. In accordance with an embodiment, the display device 44 displays three-dimensional images of the surgical site “S” which may include data captured by imaging devices (also referred to below as patient image capture devices 50) and/or include data captured by imaging devices (not shown) that are positioned about the surgical theater (e.g., an imaging device positioned within the surgical site “S”, an imaging device positioned adjacent the patient “P”, or an imaging device 52 positioned at a distal end of the imaging arm 20c). The imaging devices (e.g., imaging devices 50, 52) may capture visual images, infra-red images, ultrasound images, X-ray images, thermal images, and/or any other known real-time images of the surgical site “S” and may be cameras or endoscopes and the like. The imaging devices 50, 52 transmit captured imaging data to the controller 30 which creates three-dimensional images of the surgical site “S” in real-time from the imaging data and transmits the three-dimensional images to the display device 44 for display. In another embodiment, the displayed images are two-dimensional renderings of the data captured by the imaging devices 50, 52.

The console 40 includes input handles 43 and input devices 42 to allow a clinician to manipulate the robotic system 10 (for example, move the arms 20a, 20b, 20c, the ends 22a, 22b, 22c of the arms 20a, 20b, 20c, and/or the surgical instruments 16). Each of the input handles 43 and input devices 42 communicates with the controller 30 to transmit control signals thereto and to receive feedback signals therefrom. Additionally or alternatively, each of the input handles 43 may include control interfaces (not shown) which allow the surgeon to manipulate (for example, clamp, grasp, fire, open, close, rotate, thrust, slice, etc.) the surgical instruments 16 supported at the ends 22a, 22b, 22c of the arms 20a, 20b, 20c.

In an embodiment, the input handles 43 are moveable through a predefined workspace to move the ends 22a, 22b, 22c of the arms 20a, 20b, 20c within the surgical site “S.” It will be appreciated that while the workspace is shown in two-dimensions in FIG. 1, the workspace is a three-dimensional workspace. The three-dimensional images on the display device 44 are oriented such that movement of the input handle 43 moves the ends 22a, 22b, 22c of the arms 20a, 20b, 20c as viewed on the display device 44. It will be appreciated that the orientation of the three-dimensional images on the display device 44 may be mirrored or rotated relative to a view from above the patient “P”. In addition, it will be appreciated that the size of the three-dimensional images on the display device 44 may be scaled to be larger or smaller than the actual structures of the surgical site permitting the surgeon to have a better view of structures within the surgical site “S.” As the input handles 43 are moved, the surgical instruments 16 are moved within the surgical site “S” as detailed below. Movement of the surgical instruments 16 may also include movement of the ends 22a, 22b, 22c of the arms 20a, 20b, 20c which support the surgical instruments 16. Although illustrated as a handle, the input handle 43 may include a clutch switch and/or include gimbals and joints.

The input devices 42 are used to receive inputs from the clinician. Although depicted as a single component, more than one component may be included as part of the input devices 42. For example, multiple input devices 42 may be included as part of the console 40, and each input device 42 can be used for a different purpose. In an example, each input device 42 may be configured such that each allows the robotic surgical system 100 to enter a different operational mode. In another embodiment, the input devices 42 are configured to permit the user to make selections displayed on the display 44 (also referred to herein as “autostereoscopic display” or simply a “display”) or on a touchscreen (if included), such as from drop down menus, pop-up windows, or any other presentation mechanisms. In another embodiment, the input devices 42 are configured to permit the user to manipulate a surgical site image, such as by zooming in or out of the surgical site image, selecting a location on the surgical site image, and the like. In other embodiments, the input devices 42 may include one or multiple ones of a touchpad, joystick, keyboard, mouse, or other computer accessory, and/or a foot switch, pedal, trackball, or other actuatable device configured to translate physical movement from the clinician to signals sent to the controller 30.

The movement of the surgical instruments 16 is scaled relative to the movement of the input handles 43. When the input handles 43 are moved within a predefined workspace, the input handles 43 send control signals to the controller 30. The controller 30 analyzes the control signals to move the surgical instruments 16 in response to the control signals. The controller 30 transmits scaled control signals to the robot base 18 to move the surgical instruments 16 in response the movement of the input handles 43.

In an embodiment, the console 40 includes the user image capture device 48 (in an example, one or more cameras) to capture one or more images or videos of the user (not shown in FIG. 1). For example, the user image capture device 48 may be configured to periodically capture still images of the user, video of the user, and the like. In another embodiment, the user image capture device 48 is used to track the eyes, the face, the head or other feature(s) of the user. In an embodiment, the user image capture device 48 captures visual images, infra-red images, ultrasound images, X-ray images, thermal images, and/or any other known real-time images.

The user image capture device 48 can be integrated with, and/or positionally fixed to, the display 44, such that the positional relationship between the user image capture device 48 and the display 44 is known and can be relied upon by the controller 30 in various computations. Tracking can be enhanced with the use of a wearable 45 worn by the user to provide fixed locations in the form of markers 47 that may be detected when images of the user are processed. The wearable 45 may be provided as glasses, a headband, a set of stickers placed on locations on the user and the like. In another example, the controller 30 utilizes the images captured by the user image capture device 48 to determine a position of the user, for example, by employing a recognition and tracking algorithm that detects the markers 47 in the captured images and determines the positions of the markers 47 to obtain the position of the user. The controller 30 then compares the determined position of the user to a predetermined position criterion. In another embodiment, the controller 30 may further provide control signals based on the user's movements, allowing the movement of the user to act as an additional control mechanism for manipulating components of the robotic surgical system 100, such as the robotic arms 20a, 20b, 20c, and/or the patient image capture device 50.

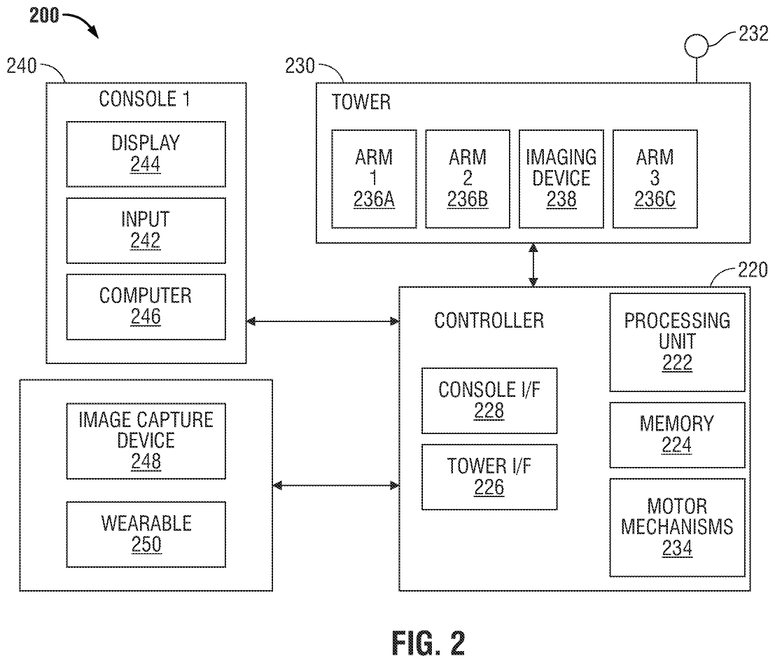

FIG. 2 is simplified block diagram of the robotic surgical system 100 of FIG. 1. The robotic surgical system 200 includes a controller 220, a tower 230, and a console 240. The controller 220 is configured to communicate with the tower 230 to thereby provide instructions for operation, in response to a signal received from the console 240.

The controller 230 generally includes a processing unit 222, a memory 224, a tower interface 226, and a consoles interface 228. The processing unit 222, in particular by means of a computer program stored in the memory 224, functions in such a way to cause components of the tower 230 to execute a desired movement of the arms 236a-c according to a movement defined by input devices 242 of the consoles 240. In this regard, the processing unit 222 includes any suitable logic control circuit adapted to perform calculations and/or operate according to a set of instructions. The processing unit 222 may include one or more processing devices, such as a microprocessor-type of processing device or other physical device capable of executing instructions stored in the memory 224 and/or processing data. The memory 224 may include transitory type memory (for example, RAM) and/or non-transitory type memory (for example, flash media, disk media, and the like). The tower interface 226 and consoles interface 228 communicate with the tower 230 and consoles 240, respectively, either wirelessly (for example, Wi-Fi, Bluetooth, LTE, and the like) and/or via wired configurations. Although depicted as separate modules, the interfaces 226, 228 are a single component in other embodiments.

The tower 230 includes a communications interface 232 configured to receive communications and/or data from the tower interface 226 for manipulating motor mechanisms 234 to thereby move arms 236a-c. In accordance with an embodiment, the motor mechanisms 234 are configured to, in response to instructions from the processing unit 222, receive an application of current for mechanical manipulation of cables (not shown) which are attached to the arms 236a-c to cause a desired movement of a selected one of the arms 236a-c and/or an instrument coupled to an arm 236a-c. The tower 230 also includes an imaging device 238, which captures real-time images of a surgical site and transmits data representing the images to the controller 230 via the communications interface 232.

To manipulate the devices of the tower 230, each console 240 has an input device 242, a display 244, and a computer 246. The input device 242 is coupled to the computer 246 and is actuated by the clinician. In this regard, the input device 242 may be one or more of a handle or pedal, or a computer accessory, such as a keyboard, joystick, mouse, button, touch screen, switch, trackball or other component. The display 244 displays images or other data received from the controller 220 to thereby communicate the data to the clinician. The computer 246 includes a processing unit and memory, which includes data, instructions and/or information related to the various components, algorithms, and/or operations of the tower 230 and can operate using any suitable electronic service, database, platform, cloud, or the like.

An image capture device 248 is included as part of the system 200 to track the movement of the user at the console 240 using, for example, a wearable 250. The image capture device 248 captures images and/or video of the user and transmits data representing the captured images and/or video to the controller 220, which is configured to process the captured images and/or video for tracking the movements of the user.

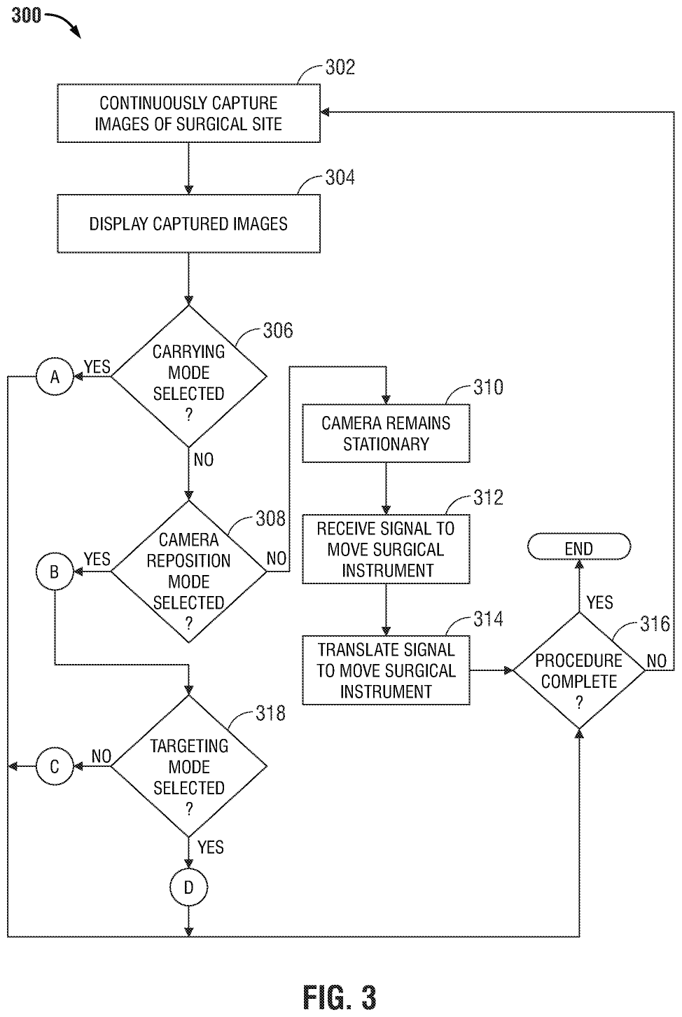

As noted above, the robotic surgical system 100, 200 may be configured to operate in one or more of a carrying mode, a camera reposition mode, and/or a targeting mode. The modes, if selected, are configured to provide one or more of permitting the clinician to cause the image capture device 50 to automatically follow the movement of a surgical instrument being used during a surgical procedure, preventing input handle 43 signals, based on actuation thereof, from affecting movement of the robotic arms 20a-c, and/or turning on a head-tracking feature. One or more of the modes may be selected during the course of operating the robotic surgical system 100, 200. FIG. 3 is a flowchart of a computer-implemented procedure 300 for operating a robotic surgical system 100, 200 having options to enter one or more of the carrying mode, the camera reposition mode and/or the targeting mode, in accordance with an embodiment. The procedure 300 may be implemented, at least in part, by the processing unit 222 executing instructions stored in the memory 224 (FIG. 2). Additionally, the particular sequence of steps shown in the procedure 300 of FIG. 3 is provided by way of example and not limitation. Thus, the steps of the procedure 300 may be executed in sequences other than the sequence shown in FIG. 3 without departing from the scope of the present disclosure. Further, some steps shown in the procedure 300 of FIG. 3 may be concurrently executed with respect to one another instead of sequentially executed with respect to one another.

As an initial matter, the clinician may activate the surgical robot 10 from the surgeon console 40 by providing an appropriate action, such as turning on the power switch, which may transmit a “power on” signal to the controller 30. In an embodiment, the clinician actuates the input handles 43 and/or input devices 42, which provide signals, based on the actuations, for selecting and manipulating one of the robotic arms 20a, 20b, or 20c for placement of the selected robotic arm 20a, 20b, or 20c at the surgical site “S.” At least one of the robotic arms 20a, 20b, or 20c includes the patient image capture device 50. In an embodiment in which a surgical instrument 16 is on a separate one of the robotic arms 20a, 20b, or 20c from that of the patient image capture device 50, the clinician actuates the input handles 43 and/or input devices 42, which provide additional signals, based on the actuation, for selecting the other robotic arm 20a, 20b, or 20c and manipulating the other selected robotic arm 20a, 20b, or 20c for placement at the surgical site “S.”

Beginning at step 302, images of the surgical site “S” are continuously captured. In this regard, the patient image capture device 50, which was placed by the clinician at a desired position within the surgical site “S”, captures visual images, infra-red images, ultrasound images, X-ray images, thermal images, and/or any other known real-time images of surgical site “S.” Data representing the images is transmitted to the controller 30, which provides commands to the display 44 to cause the captured images to be displayed at step 304. In this way, the captured images provide the clinician with a real-time view of the surgical site “S” during the performance of a surgical procedure. The captured images may include images of the tissue and one or more surgical instruments 16, such as those that have been placed in the surgical site “S” by the clinician.

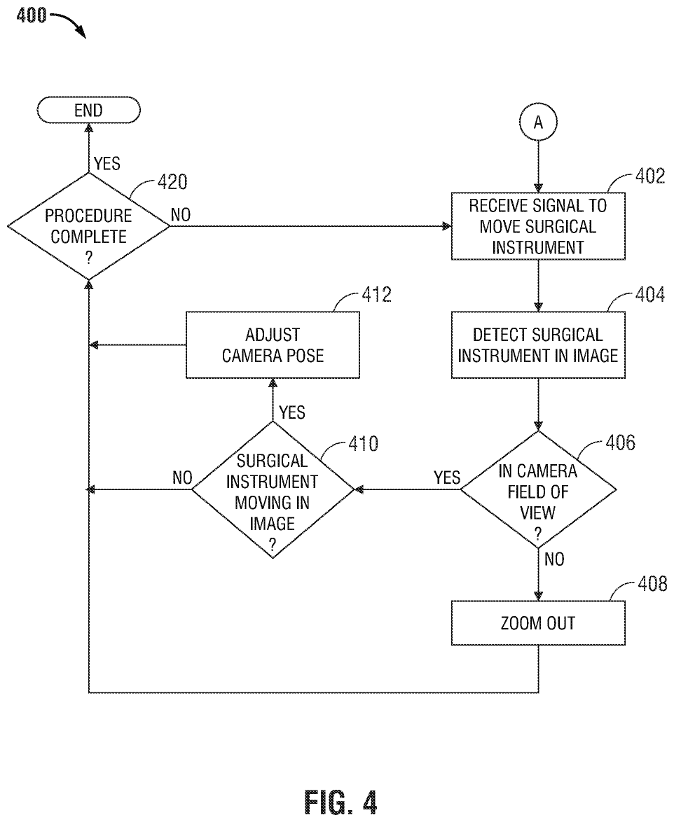

At some point during the surgical procedure, the clinician may select entry into one or more of the various modes. In particular, at step 306, the clinician may select entry into the carrying mode at step 306. Entry into the carrying mode is selected by providing a corresponding signal, based on a corresponding actuation of one of the input devices 42. For example, a command to enter or exit the carrying mode may be associated with an actuation of a foot pedal, such that a single tap of the foot pedal causes entry and/or a double tap of the foot pedal causes exit. In another example, entry into or exit from the carrying mode is associated with a sustained depression or a release of a button, a gripper, or other mechanism disposed on or adjacent the input handle 43. In still another embodiment, entry into or exit from the carrying mode is associated with a tap, a drag, or other motion across a trackpad. In response to a signal for entry into the carrying mode, the procedure 300 proceeds to process “A”, which as is discussed in more detail below in conjunction with FIG. 4, includes a method for controlling the surgical system 10 in the carrying mode. After performing process “A”, the procedure 300 continues to step 316, where a determination is made as to whether the surgical procedure is complete. If so, the procedure 300 ends. If the surgical procedure is not complete at step 316, the procedure 300 iterates at step 302.

However, if the carrying mode is not selected, for example, where no signal has been received that is associated with entering the carrying mode or a signal has been received indicating a de-selection of the carrying mode, the procedure 300 may proceed to step 308, during which the clinician may select entry into the camera reposition mode. Entry into the camera reposition mode is selected by actuating one of the input devices 42 to thereby provide a corresponding signal. It will be appreciated that entry into or exit out of the camera reposition mode may be implemented using a configuration that is different from a configuration used for implementing the carrying mode. For example, in an embodiment in which tapping the foot pedal is associated with entry into or exit from the carrying mode, depressing/releasing a button of the input handle 43 may be associated with entry into or exit from the camera reposition mode. Other associations may be employed in other embodiments.

If entry into the camera reposition mode has not been selected, for example, where no signal has been received that is associated with entering the camera reposition mode or a signal has been received indicating a de-selection of the camera reposition mode, the patient image capture device 50 remains stationary at step 310. Additionally, as the patient image capture device 50 maintains the same position in which it was placed prior to the execution of procedure 300, one or more of the input handles 43 are actuated to provide signals to move the surgical instrument 16 in the surgical site “S” at step 312, and the signals may be translated by the controller 30 to thereby effect movement of the surgical instrument 16 at step 314. A determination is made as to whether the surgical procedure is complete at step 316. If so, the procedure 300 ends. If the surgical procedure is not complete at step 316, the procedure 300 iterates at step 302.

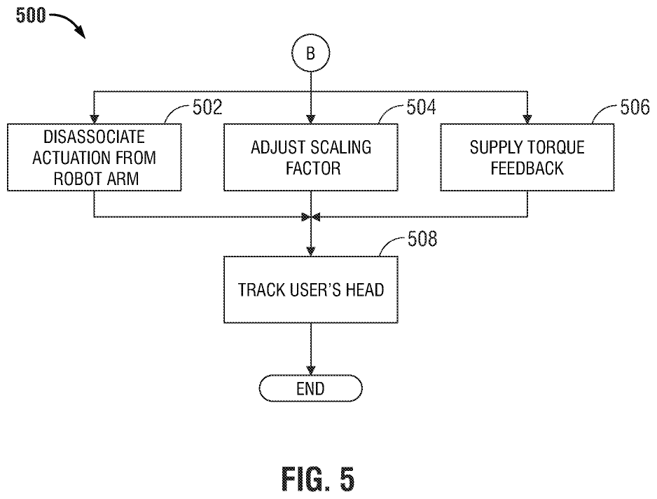

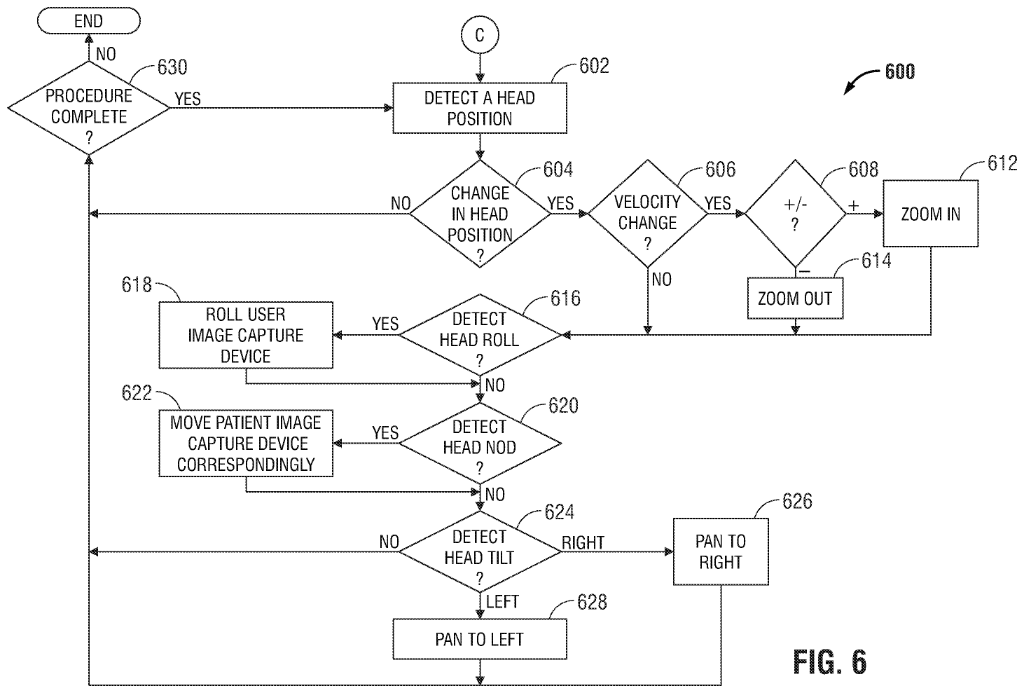

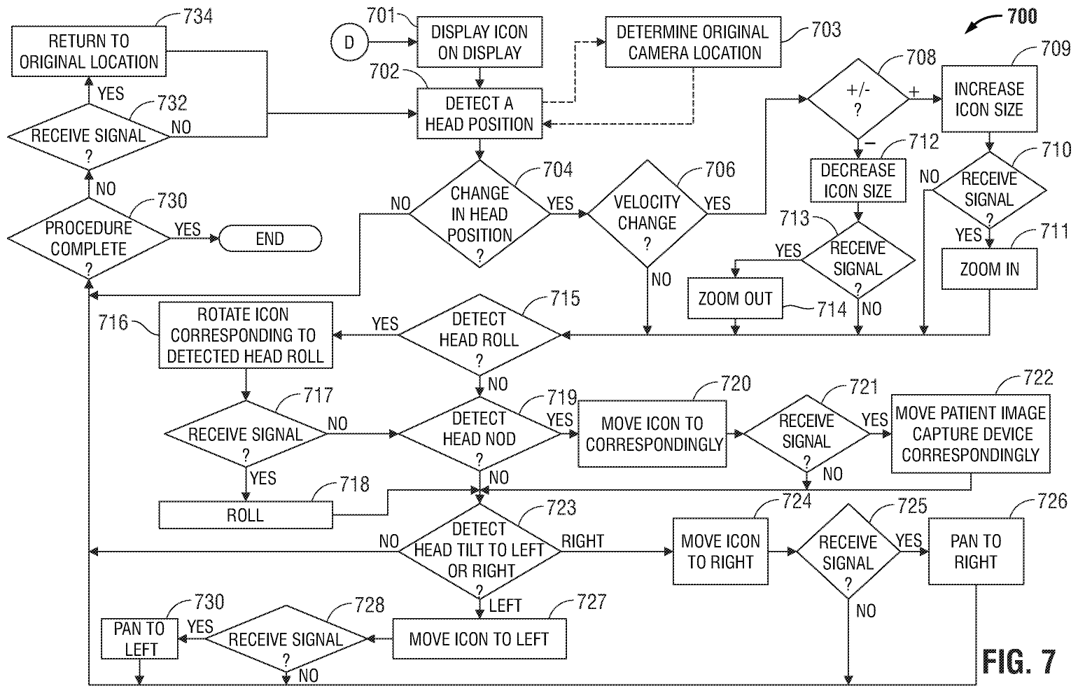

Returning to step 308, if entry into the camera reposition mode has been selected, the procedure 300 continues to process “B”, which, as is discussed below with reference to FIG. 5, includes steps for controlling the robotic surgical system in the camera reposition mode. While in the camera reposition mode, a selection for entry into the targeting mode may be made at step 318. Entry into the targeting mode is selected by providing a corresponding actuation of one of the input devices 42. As with the other modes, it will be appreciated that entry into or exit out of the target mode is implemented using a configuration that is different from the configurations used for implementing the carrying mode and the camera reposition mode. If targeting is not selected, the procedure 300 advances to process “C” of FIG. 6. If selected, the procedure continues to process “D” of FIG. 7. No matter whether entry into the targeting mode is selected or not, in each case, a determination is made as to whether the surgical procedure is complete at step 316. If so, the procedure 300 ends. If the surgical procedure is not complete at step 316, the procedure 300 iterates at step 302.

Turning now to FIG. 4, a flowchart of a computer-implemented procedure 400 for controlling the robotic surgical system when in the carrying mode will now be provided. As noted briefly above, while in the carrying mode, the image capture device 50 “follows” the surgical instrument as it is moved within the surgical site “S.” The procedure 400 may be implemented, at least in part, by the processing unit 222 executing instructions stored in the memory 224 (FIG. 2). Additionally, the particular sequence of steps shown in the procedure 400 of FIG. 4 is provided by way of example and not limitation. Thus, the steps of the procedure 400 may be executed in sequences other than the sequence shown in FIG. 4 without departing from the scope of the present disclosure. Further, some steps shown in the procedure 400 of FIG. 4 may be concurrently executed with respect to one another instead of sequentially executed with respect to one another.

Beginning at step 402, a signal, based on an actuation of an input device, is received to move the surgical instrument 16. For example, the clinician manipulates the input handles 43 to provide a signal, based on the manipulation, to move a selected one of the surgical instruments 16. In response to the received signal, the controller 30 provides commands to a corresponding robotic arm 20a, 20b, 20c to move the selected surgical instrument 16 in a corresponding manner. At step 404, substantially concurrently with the movement of the robotic arm 20a, 20b, 20c, the surgical instrument 16 is detected in images captured at step 302. For example, the movement of the surgical instrument 16 is detected from images captured at the surgical site “S,” detected by controller 30, and the like.

To determine whether a movement of the surgical instrument 16 warrants adjustment of the patient image capture device 50, at step 406 a determination is made as to whether the surgical instruments are within a field of view of the patient image capture device 50. In particular, images captured by the patient image capture device 50 are processed either optically or by image recognition to identify whether the surgical instrument 16 can be found in the image. If the surgical instrument 16 is detected within the captured image, the controller 30 provides commands to the patient image capture device 50 to determine whether the surgical instrument 16 is moving in the image at step 410. For example, the controller 30 analyzes the images over time and continuously compares the captured images to assess whether the surgical instrument 16 has moved within the image. If so, the patient image capture device 50 adjusts its pose at step 412. For example, the patient image capture device 50 adjusts its pose by turning in a direction corresponding to the movement of the surgical instrument 16 or moving to a location to permit the patient image capture device 50 to center its field of view on a predetermined location or specified identifier on the surgical instrument 16. In embodiments in which more than one surgical instrument 16 is present at the surgical site “S,” adjustments to the pose of patient image capture device 50 may depend on the locations of the one or more of surgical instruments 16 at the surgical site “S” and may be implemented by centering the field of view of the patient image capture device 50 on a designated one of the surgical instruments 16, centering the field of view of the patient image capture device 50 at a mean position of all surgical instruments 16, or centering the field of view of the patient image capture device 50 on a position according to a weighting of surgical instruments 16. The method 400 then continues to step 420 during which a determination is made as to whether the procedure is complete. If the procedure is not complete, the method 400 iterates at step 402. If the procedure is complete, the method 400 ends.

If at step 410, the surgical instrument 16 is not moving in the image, the method 400 continues to step 420 during which a determination is made as to whether the procedure is complete. If the procedure is not complete, the method 400 iterates at step 402. If the procedure is complete, the method 400 ends.

Returning to step 406, if a determination has been made that the surgical instrument 16 is outside of the field of view of the patient image capture device 50, the controller 30 provides commands to the patient image capture device 50 to decrease its focal length to thereby decrease magnification and provide a zoomed out view of the surgical site “S” at step 408 until the surgical instrument 16 is within the field of view. In either case, the method 400 continues to step 420 during which a determination is made as to whether the procedure is complete. If the procedure is not complete, the method 400 iterates at step 402. If the procedure is complete, the method 400 ends.

It is contemplated that the carrying mode may include, and not limited, to the following additional features:
Use of a motion of the master handle as the input, or the commanded instrument motion, and not measure the actual position of the instruments from image processing, wherein the system knows the relative motion of the instruments due to kinematics, so image processing would not necessarily be required;
Use of filtering schemes;
Use of nonlinear mapping of instrument motion to camera motion;
Use of mapping of motion of two (2) instruments, e.g., moving the camera based on the centroid of both instruments, or following just one or the other instrument, or some combination of both;
Use of different mapping for panning versus zoom;
Use of multiple types of carry mode that the surgeon can select (e.g., different scaling factors, filters, R/L hand tract, etc.); and/or
Use of a handle button or pedal to switch between carrying modes.

As noted briefly above, in a case in which the carrying mode has not been selected, the clinician may select entry into the camera reposition mode. The camera reposition mode permits the clinician to move the input handles 43 without effecting or minimally affecting movement of the surgical instrument 16. Such an option may be desirable because when the clinician actuates the input handles 43, the actuation may cause the input handles 43 to leave a neutral position within the surgeon console 40. Because the ratio of the movement of the input handles 43 to the surgical instrument 16 is generally small, large movements of input handles 43 result in small movements of the surgical instrument 16. However, as the input handles 43 are extended further from the neutral position, it becomes more difficult and more uncomfortable for the clinician to manipulate the input handles 43. Accordingly, from time to time, the clinician resets the positioning of the input handles 43 to a more centralized position to continue performing the procedure.

With reference to FIG. 5, a flowchart of a computer-implemented procedure 500 for operating a robotic surgical system 10, in accordance with another embodiment is provided. The procedure 500 may be implemented, at least in part, by the processing unit 222 executing instructions stored in the memory 224 (FIG. 2). Additionally, the particular sequence of steps shown in the procedure 500 of FIG. 5 is provided by way of example and not limitation. Thus, the steps of the procedure 500 may be executed in sequences other than the sequence shown in FIG. 5 without departing from the scope of the present disclosure. Further, some steps shown in the procedure 500 of FIG. 5 may be concurrently executed with respect to one another instead of sequentially executed with respect to one another.

At step 502, in response to the received mode selection, commands are provided to disassociate actuation of the input handles 43 from movement of the robotic arms 20a, 20b, 20c. The selection may be received, for example, by actuation of one of the input devices 42, such as through a foot pedal. In an embodiment, a signal received based on the actuation indicating that the foot pedal has been depressed or a signal received based on the actuation indicating that the foot pedal has been released, may be received by the controller 30. According to an embodiment, the controller 30, in response to the received mode selection, provides a disassociate command and initiates a protocol to disassociate actuation of the input handles 43 from movement of the robotic arms 20a, 20b, 20c. In an embodiment, disassociation occurs by providing a command to cause a gear association within the motor 18 to disengage by altering the location of the gear so that gears associated with movement of input handles 43 continue to rotate but do not contact or engage gears associated with movement of robotic arms 20a, 20b, 20c. In another embodiment, signals resulting from the received input at the input handles 43 are received by the controller 30, but are not delivered to the robotic arms 20a, 20b, 20c thereby preventing movement of the robotic arms 20a, 20b, 20c despite the received input.

In addition or as an alternative to step 504, in response to the received mode selection, commands are provided to adjust a scaling factor between the movement of the input handles 43 and the movement of the robotic arms 20a, 20b, 20c and/or surgical instruments 16. For example, when the clinician actuates the input handle 43, a signal, based on the actuation, is translated by the controller 30 into a motion. Joint angles of the input handle 43 are measured from the signal to allow forward kinematics of the signal to be obtained, and based on the pose of the input handle 43, scaling and clutching are applied to the pose of the input handle 43 to output a desired pose for the robotic arms 20a, 20b, 20c and/or surgical instruments 16. In an example, during normal operation, a scaling factor between the movement of the input handles 43 and the movement of the robotic arms and/or instrument may be 10:1 (e.g., a 10 mm movement of the input handle 43 causes a 1 mm movement of the robotic arm robotic arms and/or instrument). When the scaling factor is adjusted as a result of step 504, the scaling factor may be adjusted so that a greater movement of the input handles 43 is needed in order to effect movement of the robotic arms and/or instrument (e.g., a 10 mm movement of the input handle 34 causes a 0.0001 mm movement of the robotic arms and/or instrument). As result, the robotic arms 20 and consequently surgical instruments 16 move at nearly imperceptible amounts in response to the actuation of the input handle 43, allowing the clinician to move the input handles 43 to more easily move the input handles 43 into a more comfortable position.

In addition or as an alternative to steps 504 and 506, in response to the received mode selection, a torque feedback is supplied at step 508. For example, when the clinician actuates the input handle 43, a signal, based on the actuation, is translated by the controller 30 into a motion. Joint angles of the input handle 43 are measured from the signal to allow forward kinematics of the input to be obtained, and based on the pose of the input handle 43 scaling and clutching are applied, if desired, to the pose of the input handle 43 to output a desired pose for the robotic arms and/or instrument. A force/torque feedback wrench is calculated based on actual slave joint angles output by the robotic arms and/or instrument. The force/torque feedback of the slave joint limits, velocity limits, and collisions may be stored in memory and hence, pre-set by the expert clinician, depending on the expert clinician's preference or may be included as a factory-installed parameter. The force/torque command (F/T wrench) output is processed using a transpose Jacobian function to calculate the required joint torques in the input device to output the desired slave wrench commands, and the required input device joint torques are then combined with the joint torques required for hold/reposition modes and range of motion limits, which may be predetermined values that are provided in response to the received mode selection, and gravity and friction compensation (if desired). In any case, as a result of the combination, the joint torques for the input handle 43 are obtained and taken into account when the clinician actuates the handles 43 so that when the additional movements are received, the controller 30 causes the motor to output a force that is equal to and opposite the input force, thereby canceling the movement of the robotic arms and/or instrument despite the clinician's actuation of the input handles 43.

In addition to the performance of one or more of the disassociation steps, the procedure 500 includes tracking a user's head at step 508. In an embodiment, the user's head is tracked via the user image capture device 48, which is directed at and captures images of the user and the markers 27. Specifically, captured images of the user are processed by the controller 30 and markers 27 may be isolated from the captured images and tracked over time. For example, the markers 27 include one or more infrared markers (not shown) perceptible by the user image capture device 48, which images the one or more infrared indicators and provides image data to the controller 30. The controller 30 processes the images provided by user image capture device 48 and determines the locations of the one or more infrared markers in a 2-dimensional plane. Movement of the user's head is detected by processing changes in the locations of the one or more infrared markers over time. By tracking movement of the one or more markers 27 on the wearable 45, for example, attached to the head of the clinician operating the robotic system 100, the controller 30 tracks the motion of the user's head.

The head movements detected during the head tracking of step 508 may be used to provide signals to the system 100, which when received cause the controller 30 to provide commands to the patient image capture device 50 to alter its pose and/or to zoom in or out to capture desired images for display on the display 44. For example, in an embodiment in which the targeting mode is not selected, the tracked head movements are used to directly drive the movement of the patient image capture device 50. An example of a computer-implemented procedure 600 for controlling the robotic surgical system when not in the targeting mode is provided in FIG. 6, in accordance with an embodiment. The procedure 600 may be implemented, at least in part, by the processing unit 222 executing instructions stored in the memory 224 (FIG. 2). Additionally, the particular sequence of steps shown in the procedure 600 of FIG. 6 is provided by way of example and not limitation. Thus, the steps of the procedure 600 may be executed in sequences other than the sequence shown in FIG. 6 without departing from the scope of the present disclosure. Further, some steps shown in the procedure 600 of FIG. 6 may be concurrently executed with respect to one another instead of sequentially executed with respect to one another.

In an embodiment, a head position is detected at step 602. The head position is determined from the captured images of the user from the head tracking of step 508 (FIG. 5) and is performed in a manner similar to one described above with respect of step 508 of FIG. 5. In an embodiment, an initial head position is detected.

Next, a determination is made as to whether the head position of the user has changed at step 604. More particularly, as the images are captured over time by the user image capture device 48, the controller 30 continuously processes the image to detect the markers 27, determines positions of the markers 27 over a period of time, and calculates the centroid of the detected markers 27 over the period of time. If the centroid has not changed position, the procedure continues to step 630, where a determination is made as to whether the surgical procedure is complete. If so, the procedure 600 ends. If not, the procedure 600 iterates at step 602. However, if the centroid changes position, a determination is made that the user's head position has changed. In response to a detected change in position, a determination is made as to whether the change in head position is a velocity change at step 606. If so, a determination is then made as to whether the velocity includes a positive or negative change at step 608.

If the velocity indicates a positive change, a determination is made that the clinician's head is moving in a forward direction toward the user image capture device 48, and the controller 30 provides commands to the patient image capture device 50 to correspondingly magnify the captured images of the surgical site “S” at step 612. Likewise, if the velocity indicates a negative change, a determination is made that the clinician's head is moving in a backward direction away from the user image capture device 48, and the controller 30 provides commands to the patient image capture device 50 to correspondingly zoom out from the surgical site “S” at step 614. In an embodiment, the amount of magnification, effected by the focal length of the lens within the patient image capture device 50, is directly proportional to the head movement in the forward or backward directions. In another embodiment, the amount of magnification is scaled relative to the head movement in the forward or backward directions.

In another embodiment of steps 612 and 614, the operations are implemented in a different manner. For example, in accordance with another embodiment, a distance between the user's head position from a plane of the display 44 is determined from the velocity and the detected location of the markers 27, and the distance is used to determine a magnification used by the patient image capture device 50 in the capturing of the images of the surgical site “S.” In this embodiment, the memory 224 may have stored thereon a database including head position distances from the display 44 and corresponding magnification amounts, so that the controller 30 may refer to the database during step 608 in its determination of whether (and how much) to zoom in or out. In another embodiment, a size of the markers 27 detected in the captured images is used to determine the distance of the user's head position from the plane of the display 44. Thus, in response to a detection that the markers 27 are becoming progressively larger in size over a period of time, a determination is made that the user's head is moving in the forward direction. Likewise, in response to a detection that the markers 27 are becoming progressively smaller in size over a period of time, a determination is made that the user's head is moving in the backward direction. After either of steps 612 or 614, the procedure 600 continues to step 616. Additionally, in an embodiment in which no velocity change is detected at step 606, the procedure 600 also continues to step 616.

At step 616, a determination is made as to whether a head roll has been detected. A head roll may be performed by the clinician either on the right side by moving the clinician's right ear closer to the clinician's right shoulder or on the left side by moving the clinician's left ear closer to the clinician's left shoulder. In an embodiment, the head roll may be associated with a command for rotating a patient image capture device 50 having an angled neck, such as a 30° endoscope. In an example, as the images are captured over time by the user image capture device 48, the controller 30 continuously processes the images to detect the markers 27 and determines the positions of the markers 27. Based on the positions of the markers 27, the controller 30 can determine whether a head roll has been detected. For example, in an embodiment in which two or more markers are included, the two or more markers rotate in a clockwise motion to indicate a head roll to the right side or in a counter-clockwise motion to indicate a head roll to the left side. In response to the detection of a head roll, the controller 30 provides commands to the patient image capture device 50 to roll as well at step 618. In particular, the patient image capture device 50 rotates in a clockwise motion, in response to the head roll being performed on the right side, or rotates in a counter-clockwise motion, in response to the head roll being performed on the left side. The amount the patient image capture device 50 is rotated may be directly proportional, scaled relative to the movement of the head roll, or rotated based on some other mathematical function. The procedure 600 proceeds to step 620. Similarly, in an embodiment in which a head roll has not been detected, the procedure 600 continues to step 620.

At step 620, a determination is made as to whether a head nod has been detected. A head nod may be performed by the clinician by moving the clinician's chin in an up and down direction. In an example, as the images are captured over time by the user image capture device 48, the controller 30 continuously processes the images to detect the markers 27 and determines the positions of the markers 27. Based on the positions of the markers 27, the controller 30 can determine whether a head nod has been detected. For example, the markers 27 may change position along a y-axis of the image, and in response, the controller 30 may determine that the clinician is performing a head nod. In response to the detected head nod, the controller 30 provides commands to the patient image capture device 50 to adjust its view of the surgical site “S” in a corresponding manner at step 622. For example, in response to the markers 27 being detected as moving upwards along the y-axis of the image, the controller 30 provides commands to move the patient image capture device 50 in the same manner. The procedure 600 proceeds to step 624. Similarly, in an embodiment in which a head nod has not been detected, the procedure 600 continues to step 624.

At step 624, a determination is made as to whether a head tilt has been detected. A head tilt may be performed by the clinician by moving the clinician's head either to the left or right, similar to a head shaking motion. In an example, as the images are captured over time by the user image capture device 48, the controller 30 continuously processes the images to detect the markers 27 and determines the positions of the markers 27. Based on the positions of the markers 27, the controller 30 can determine whether a head tilt has been detected. For example, the markers 27 may change position along an x-axis of the image, and in response, the controller 30 may determine that the clinician is performing a head tilt. In response to the detected head tilt, the controller 30 provides commands to the patient image capture device 50 to pan right, if the direction of the head tilt is toward the right at step 626, or to pan left, if the direction of the head tile is toward the left at step 628. Steps 626 and 628 are implemented by mapping the velocity of the head position to the velocity of the patient image capture device 50, in an embodiment. Returning to step 624, if a head tilt has not been detected, the procedure continues to step 630, where a determination is made as to whether the surgical procedure is complete. If so, the procedure 600 ends. If not, the procedure 600 iterates at step 602. While head rolling, tilting and nodding have been described in detail herein, it is further contemplated that the system according to the present disclosure can accommodate and adjust for translational motion of the head relative to the shoulders (e.g., sliding of the head side-to-side along an axis parallel to an axis defined by the shoulders, or sliding of the head up-and-down/forward-and-back along an axis perpendicular to an axis defined by the shoulders).

In another embodiment of using head movements detected during the head tracking of step 508, the clinician may select entry into the targeting mode. As noted briefly above, the targeting mode permits the clinician to drive an icon displayed on the display 44 and to provide a selection, via actuation of a button, foot pedal, touch pad or other input device, to confirm the desire for a movement of the patient image capture device 50. An example of a computer-implemented procedure 700 for controlling the robotic surgical system when in the targeting mode is provided in FIG. 7, in accordance with an embodiment. The procedure 700 may be implemented, at least in part, by the processing unit 222 executing instructions stored in the memory 224 (FIG. 2). Additionally, the particular sequence of steps shown in the procedure 700 of FIG. 7 is provided by way of example and not limitation. Thus, the steps of the procedure 700 may be executed in sequences other than the sequence shown in FIG. 7 without departing from the scope of the present disclosure. Further, some steps shown in the procedure 700 of FIG. 7 may be concurrently executed with respect to one another instead of sequentially executed with respect to one another.

In an embodiment, an icon is displayed on a display at step 701. For example, the icon is superimposed over the image of the surgical site “S” captured by the patient image capture device 50 and may be a two-dimensional or three-dimensional shape, such as a triangle, prism, or other suitable representation which when viewed by the clinician can indicate direction. The icon may be displayed in the center of the image to indicate a center of the field of view of the patient image capture device 50. In addition to displaying the icon, the user's head position is detected at step 702. The head position is determined from the captured images of the user from the head tracking of step 508 (FIG. 5) and is performed in a manner similar to one described above with respect to step 508 of FIG. 5. In an embodiment, an initial head position is detected. Optionally, an initial location of the patient image capture device 50 is obtained at step 703. The obtained initial location is stored in the memory 224 for later use.

At step 704, a determination is made as to whether the head position of the user has changed. For example, as the images are captured over time by the user image capture device 48, the controller 30 continuously processes the images to detect the markers 27 in the images, determines positions of the markers 27 over a period of time, and calculates the centroid of the detected markers 27 over the period of time. If the centroid has not changed position, the procedure continues to step 730, where a determination is made as to whether the surgical procedure is complete. If so, the procedure 700 ends. If not, the procedure 700 proceeds to step 732 where a determination is made as to whether signal, based on an actuation, has been received to return the patient image capture device 50 to the initial location. As such, the controller 30 obtains the initial location from the memory 224 and provides commands to the patient image capture device 50 to return to the initial location at step 734. If no signal has been received, the procedure 700 iterates at step 702.

If the centroid changes position, a determination is made that the user's head position has changed and in response, a determination is made as to whether a velocity of the head position has changed at step 706. If so, a determination is then made as to whether the velocity includes a positive or negative change at step 708.

In an embodiment in which the velocity indicates a positive change, which is associated with the clinician's head moving in a forward direction, the controller 30 provides commands to increase the size of the icon in a manner corresponding to the forward movement at step 709. A determination is then made as to whether a signal, based on an actuation of one or more of the input devices 42, has been received to indicate a confirmation by the clinician to cause the patient image capture device 50 to correspondingly magnify the captured images of the surgical site “S” at step 710. In an embodiment, the clinician presses or releases a user input device, such as a button, a foot pedal, a touch screen, and the like. In another embodiment, the controller 30 determines that a confirmatory signal, based on the actuation, has been received, by, for example, a timer expiring. In response to the signal being received, the controller 30 provides commands to the patient image capture device 50 to correspondingly magnify or zoom into the captured images of the surgical site “S” at step 711. Likewise, if the velocity indicates a negative change indicating that the clinician's head is moving in a backward direction, the controller 30 provides commands to the patient image capture device 50 to decrease the size of the icon in a manner correspond to the backward movement at step 712. A determination is made as to whether a signal has been received to indicate a confirmation by the clinician to cause the patient image capture device 50 to correspondingly de-magnify the captured images of the surgical site “S” at step 713. In response to the signal being received, the controller 30 provides commands to the patient image capture device 50 to correspondingly zoom out from the surgical site “S” at step 714. As with the embodiment described in conjunction with the procedure 600, the amount of magnification, effected by the focal length of the lens within the patient image capture device 50, may be directly proportional to the head movement in the forward or backward directions, the amount of magnification is scaled relative to the head movement in the forward or backward directions, or the like. Additionally, steps 712 and 714 may be implemented in a manner similar to that described above with respect to steps 612 and 614 of procedure 600, respectively.

After either of steps 712 or 714, the procedure 700 continues to step 715. Additionally, in an embodiment in which no velocity change in head position is detected at step 706, the procedure 700 also continues to step 715.

At step 715, a determination is made as to whether a head roll has been detected. As the images are captured over time by the user image capture device 48, the controller 30 continuously processes the image to detect the markers 27 and determines the positions of the markers 27. Based on the positions of the markers 27, the controller 30 determines whether a head roll has been detected by determining whether the markers 27 have rotated in a clockwise motion to indicate a head roll to the right side or in a counter-clockwise motion to indicate a head roll to the left side. In response to the detection of a head roll, the controller 30 provides commands causing the icon rotate in a manner corresponding to the detected head roll at step 716. In an example, in response to the markers 27 rotating in a clockwise motion, commands are provided to rotate the icon on the display 44 in a clockwise motion. Similarly, in response to the markers 27 rotating in a counter-clockwise motion, commands are provided to rotate the icon on the display 44 in a counter-clockwise motion.

A determination is made as to whether a signal has been received to indicate a confirmation by the clinician to cause the patient image capture device 50 to correspondingly roll at step 717. In response to the determination indicating a confirmation signal, the controller 30 provides commands to the patient image capture device 50 to roll as well at step 718. In particular, the patient image capture device 50 rotates in a clockwise motion, in response to the head roll being performed in a right side motion, or rotates in a counter-clockwise motion, in response to the head roll being performed in a left side motion. The procedure 700 proceeds to step 719. Similarly, in an embodiment in which a head roll has not been detected, the procedure 700 continues to step 719.

At step 719, a determination is made as to whether a head nod has been detected. For example, as the images are captured over time by the user image capture device 48, the controller 30 continuously processes the images to detect the markers 27 and determines the positions of the markers 27. Based on the positions of the markers 27, the controller 30 can determine whether a head nod has been detected, such as by determining whether the markers 27 have changed position along a y-axis of the image. In response to detecting the head nod, the controller 30 may provide commands causing the icon on the display 44 to move in a manner corresponding to the head nod at step 720, such as along the y-axis of the image. A determination is made as to whether a signal has been received to indicate a confirmation by the clinician to cause the patient image capture device 50 to move in a manner corresponding to the movement of the head nod at step 721. In response to receiving the signal, the controller 30 provides commands to the patient image capture device 50 to adjust its view of the surgical site “S” in a corresponding manner at step 722. The procedure 700 proceeds to step 723. In an embodiment in which a head nod has not been detected, the procedure 700 continues to step 723.

At step 723, a determination is made as to whether a head tilt has been detected. As the images are captured over time by the user image capture device 48, the controller 30 continuously processes the images to detect the markers 27 and determines the positions of the markers 27. Based on the positions of the markers 27, for example, along an x-axis of the image, the controller 30 can determine whether a head tilt has been detected. In response to the detected head tilt, the controller 30 provides commands to cause the icon to move across the image in a corresponding manner. For example, in response to the head tilt being detected as toward the right, the controller 30 provides commands to move the icon across the image toward the right at step 724. A determination is made as to whether a signal has been received to indicate a confirmation by the clinician to cause the patient image capture device 50 to move in a manner corresponding to the head tilt at step 725. In response to the received signal, the controller 30 provides commands to the patient image capture device 50 to pan right at step 726.

Returning to step 723, in response to the detected head tilt being toward the left, the controller 30 provides commands to move the icon across the image toward the left at step 727. A determination is made as to whether a signal has been received to indicate a confirmation by the clinician to cause the patient image capture device 50 to move in a manner corresponding to the head tilt at step 728. In response to the received signal, the controller 30 provides commands to the patient image capture device to pan left at step 729. Returning to step 723, if a head tilt has not been detected, the procedure continues to step 730, where a determination is made as to whether the surgical procedure is complete. If so, the procedure 700 ends. If not, the procedure 700 continues to step 732, as described above.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A robotic surgical system, comprising:
a robotic arm including a surgical instrument;
a patient image capture device configured to capture images of a surgical site;
a console including:
a display for displaying the captured images of the surgical site,
an input handle, and
an input device configured to be actuated and to provide a signal based on the actuation for causing the robotic surgical system to enter or exit a camera reposition mode; and
a controller coupled to the robotic arm, the patient image capture device, and the console, the controller including:
a processor, and
memory coupled to the processor and having instructions stored thereon that, when executed by the processor, cause the controller to:
in response to the signal received based on actuation of the input device, cause the robotic surgical system to enter the camera reposition mode, and
when the robotic surgical system is in the camera reposition mode, disassociate actuation of the input handle from movement of the robotic arm, and
track a position of a user's head.

2. The robotic surgical system of claim 1, wherein the input device includes a button on the input handle.

3. The robotic surgical system of claim 1, wherein the input device includes a foot pedal.

4. The robotic surgical system of claim 3, wherein the memory has further instructions stored thereon that, when executed by the processor, cause the controller to:
enter the camera reposition mode in response to receiving a first signal based on a first actuation of the foot pedal, and
exit the camera reposition mode in response to receiving a second signal based on a second actuation of the foot pedal within a predetermined time of the receiving of the first signal.

5. The robotic surgical system of claim 3, wherein the memory has further instructions stored thereon that, when executed by the processor, cause the controller to:
enter the camera reposition mode in response to receiving a signal indicating that the foot pedal has been depressed, and
exit the camera reposition mode in response to receiving a signal indicating that the foot pedal has been released.

6. The robotic surgical system of claim 1, further comprising:
a user image capture device configured to capture images of the user for tracking a motion of the user's head.

7. The robotic surgical system of claim 6, wherein the memory has further instructions stored thereon that, when executed by the processor, cause the controller to:
detect the position of the user's head from images of the user captured by the user image capture device;
determine from the captured images of the user whether a left or right tilt of the user's head has occurred; and
in response to a determination that the tilt of the user's head is a left tilt or a right tilt, cause the patient image capture device to correspondingly pan to the left or to the right.

8. The robotic surgical system of claim 6, wherein the memory has further instructions stored thereon that, when executed by the processor, cause the controller to:
detect the position of the user's head from the captured images of the user;
determine whether a roll of the user's head has occurred; and
in response to the determination of the roll of the user's head, cause the patient image capture device to roll in a motion corresponding to the roll of the user's head.

9. The robotic surgical system of claim 1, wherein the memory has further instructions stored thereon that, when executed by the processor, cause the controller to, when in the camera reposition mode, increase a scaling factor between a signal received based on actuation of the input handle and an output movement by the surgical instrument.

10. The robotic surgical system of claim 1, wherein the memory has further instructions stored thereon that, when executed by the processor, cause the controller to, when the robotic surgical system is in the camera reposition mode, provide at least one of a force feedback signal or a torque feedback signal to reduce an output movement by the surgical instrument corresponding to the signal received based on the actuation of the input handle to prevent manipulation of the input handle from moving the surgical instrument.

11-57. (canceled)
